(19) United States
(12) Patent Application Publication
(10) Pub. No.: US 2007/0116734 A1
(43) Pub. Date: May 24, 2007

(54) POROUS, LOAD-BEARING, CERAMIC OR METAL IMPLANT

(76) Inventor: Akash Akash, Salt lake City, UT (US)

Correspondence Address: CERAMATEC, INC., 2425 SOUTH 900 WEST, SALT LAKE CITY, UT, 84119, US

(21) Appl. No.: 11/561,856

(22) Filed: Nov. 20, 2006

Related U.S. Application Data
(60) Provisional application No. 60/738,202, filed on Nov. 18, 2005.

(51) Int. Cl. A61F 2/28; A61F 2/02; B29C 65/00; C04B 33/36

(52) U.S. Cl. 424/423; 623/23.5; 264/44; 623/23.74; 264/629

(57) ABSTRACT

A method and apparatus for adjusting the modulus of elasticity, flexural strength, or porosity of metal and ceramic implants is disclosed in one embodiment of the invention as including a green tape comprising metal or ceramic particles, or a combination thereof, for incorporation into a solid implant structure. Apertures are cut in selected regions of the green tape in order to create a desired pore structure in the solid implant structure. This pore structure may be designed to give the solid structure a desired modulus of elasticity, flexural strength, or porosity as well as to promote bone ingrowth. The green tape may then be layered in an orientation that will provide the desired pore structure and the metal or ceramic particles and layers may be fused together to create the solid implant structure.

### RELATED APPLICATIONS

This application claims priority to U.S. Provisional Patent No. 60/738,202 filed on Nov. 18, 2005 and entitled POROUS METAL AND CERAMIC IMPLANTS FOR LOAD BEARING APPLICATIONS AND DRUG DELIVERY.

### BACKGROUND OF THE INVENTION

1. Field of the Invention

This invention relates to medical implants and more particularly to porous load-bearing implants for use with humans and animals.

2. Description of the Relayed Art

Metal and ceramic implants are widely used to replace missing or damaged biological structures, such as bone or tissue. One problem with current orthopedic implants, particularly with metal hip stem implants, is the large difference in the modulus of elasticity of the metal implant compared to that of the cortical bone into which it is implanted. The much stiffer metal tends to bear the majority of the stresses applied to the hip, producing a “stress shielding” effect. This leaves the bone comparatively unstressed, causing it to deteriorate and resorb into the body, a process known as “disuse atrophy.” This condition also weakens the interface between the implant and the bone, resulting in aseptic loosening. In addition to the significant pain this may produce, this condition may eventually create a need for painful and costly revision surgery.

To make the modulus of elasticity of an implant closer to that of bone, the use of foamed metal has been suggested as one possible solution. Foamed metals, compared to their solid counterparts, typically have a reduced modulus of elasticity as a result of their porous structure. This modulus of elasticity will typically continue to decrease as the metal's porosity increases. Nevertheless, one of the undesirable properties of foamed metals or other porous materials is the reduction in strength and increase in brittleness that may occur as porosity increases. This decreased strength makes porous materials a relatively poor candidate for use in load-bearing implants, such as hip stem implants.

In view of the foregoing, what are needed are porous metal and ceramic load-bearing implants that have a reduced modulus of elasticity while still retaining the strength necessary for load-bearing applications. Further needed are systems and methods for precisely designing the pore structure of such implants to achieve a desired strength and flexibility. Ideally, by properly designing the pore structure, an implant could be engineered to mimic the strength, stiffness, and modulus of natural bone. Such a pore structure may be further designed to promote bone ingrowth or deliver beneficial agents such as bone growth factors or other medicaments around the implant.

### SUMMARY OF THE INVENTION

The present invention has been developed in response to the present state of the art, and in particular, in response to the problems and needs in the art that have not yet been fully solved by currently available apparatus and methods. Accordingly, an apparatus and method has been developed for processing an implant. In one embodiment, the process allows the adjusting of the modulus of elasticity of metal and ceramic implants to more closely mimic that of natural bone.

In one embodiment in accordance with the invention, a method for processing an implant and adjusting the modulus of elasticity, flexural strength, or porosity of metal and ceramic implants includes providing a green tape comprising metal or ceramic particles, or a combination thereof, for incorporation into a solid implant structure. Apertures are cut in selected regions of the green tape in order to create a desired pore or aperture structure in the solid implant structure. This pore structure may be designed to give the solid implant structure a desired modulus of elasticity. The green tape may then be layered in an orientation that will provide the desired pore structure and the metal and/or ceramic particles and layers may be fused together to create the solid implant structure.

In selected embodiments, the apertures may be cut in the green tape by laser cutting, etching, mechanical cutting, burning, or the like. In selected embodiments, the apertures may be elongated apertures. The elongated apertures may, in certain embodiments, be oriented to have a desired directional anisotropy. Similarly the pore structure created by the apertures may include a network of interconnected pores or closed pores. In certain embodiments, the pore structure may be characterized by a pore density that varies between an outer surface and a core of the solid implant structure.

To fuse the ceramic and/or metal particles together to create a solid implant structure, the method may also include pressing the layered green tape together to form a laminated structure, firing the laminated structure to burn off organic materials in the laminated structure, and sintering the laminated structure. If desired, the pore structure of the resulting solid implant structure may be infiltrated with beneficial agents to assist with bone ingrowth, healing, osteoconduction, osteointegration, drug delivery, or the like.

In another aspect of the invention, an implant in accordance with the invention may include a solid implant structure having multiple layers fused together. These layers include metal or ceramic particles, or a combination thereof, fused together. The layers are provided with apertures cut therein to provide a desired pore structure in the solid implant structure. This pore structure may be designed to provide a desired modulus of elasticity to the solid implant structure.

In certain embodiments, the apertures may be elongated apertures. These elongated apertures may or may not be oriented to have a desired directional anisotropy. Similarly, the pore structure created by the apertures may include a network of interconnected or closed pore. These pores may be characterized by a pore density that varies between an outer surface and a core of the solid implant structure. In selected embodiments, these pores may be infiltrated with beneficial agents for delivery to the body around the implant.

The present invention relates to apparatus and methods for adjusting the modulus of elasticity of metal and ceramic implants to more closely mimic that of natural bone. The features and advantages of the present invention will become more fully apparent from the following description and appended claims, or may be learned by the practice of the invention as set forth hereinafter.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through the use of the accompanying drawings, in which:

FIG. 1A is a perspective view of one embodiment of a porous hip implant characterized by an improved modulus of elasticity;

FIG. 1B is a cross-sectional view of the porous hip implant illustrated in FIG. 1A;

FIGS. 2A through 2I show various aperture shapes and patterns that maybe used to produce an implant in accordance with the invention;

FIG. 3 is a perspective view showing one way to stack various layers of green tape together to produce an implant in accordance with the invention;

FIGS. 4A through 4E represent various profile views of a layered implant structure used to produce an implant in accordance with the invention; and

FIG. 5 is a flow diagram of one embodiment of a method for producing an implant in accordance with the invention.

### DETAILED DESCRIPTION OF THE INVENTION

It will be readily understood that the components of the present invention, as generally described and illustrated in the Figures herein, could be arranged and designed in a wide variety of different configurations. Thus, the following more detailed description of the embodiments of apparatus in accordance with the present invention, as represented in the Figures, is not intended to limit the scope of the invention, as claimed, but is merely representative of certain examples of presently contemplated embodiments in accordance with the invention. The presently described embodiments will be best understood by reference to the drawings, wherein like parts are designated by like numerals throughout.

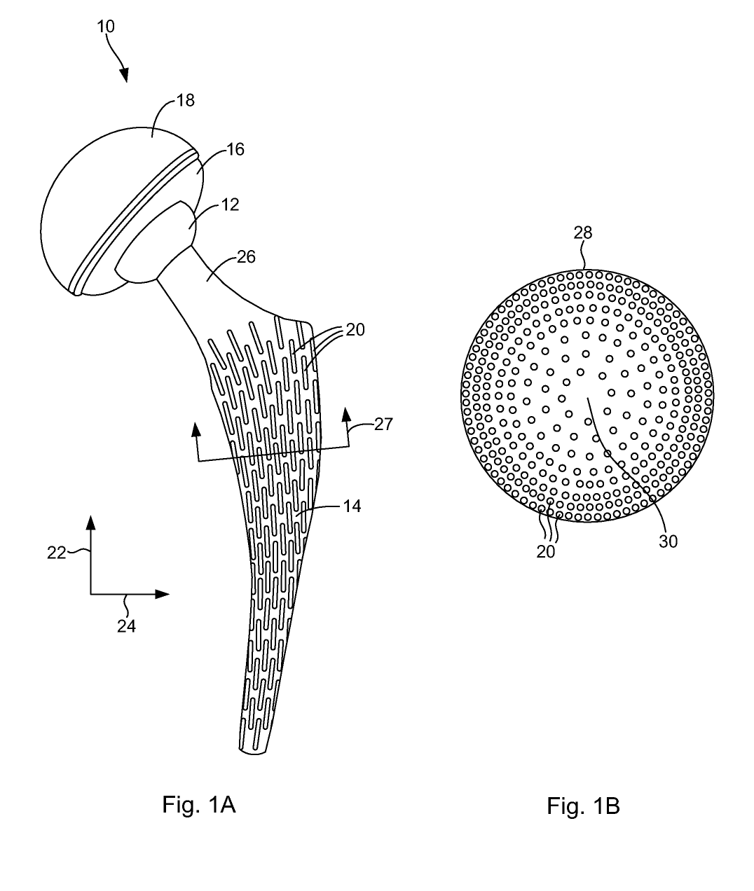

Referring to FIG. 1A, one embodiment of a porous implant 10 in accordance with the invention, in this example a hip implant 10, is illustrated. Although particular reference is made herein to a hip implant 10, the principles taught herein may be readily applied to other types of load-bearing or non-load-bearing implants. Thus, the illustrated implant 10 is simply one of many possible embodiments of an implant 10 that may take advantage of the apparatus and methods disclosed herein.

As shown, one embodiment of a hip implant 10 for performing hip replacement surgery may include a hip ball 12 connected to a stem 14. The hip ball 12 may fit into a liner 16 which may in turn be inserted into a metal shell 18 which is anchored to a recipient's pelvis. The stem 14, on the other hand, is inserted into the recipient's femur. The hip ball 12 is typically constructed of metal or ceramic while the liner 16 may be typically made of polyethylene, metal, or ceramic.

To provide sufficient strength, the stem 14 is typically constructed of metal. As previously mentioned, one problem with this metal construction is the large difference in modulus of elasticity of the metal compared to that of cortical bone. This creates a “stress shielding” effect that leaves the bone comparatively unstressed, causing it to deteriorate and resorb into the body. This condition may result in aseptic loosening by weakening the interface between the implant 10 and the bone. Another disadvantage of metal implants is their tendency to reflect x-rays or radio waves, which impairs postoperative radiographic evaluation of the implant 10.

To modify the stem's modulus of elasticity to more closely mimic that of natural bone, a pore structure 20 may be incorporated into the implant 10 to change the stem's modulus of elasticity. Unlike the random pore structure of foamed materials, this pore structure 20 may be carefully designed to retain much of the strength required by the implant while still improving the implant's modulus of elasticity. Ideally, this will reduce stress shielding and aseptic loosening that is characteristic of many current implants. Furthermore, by properly sizing the pores 20, the pore structure may also provide an improved bond between the stem 14 and the femur by promoting bone ingrowth into the pores 10. In certain embodiments, pores 20 having a size between about 0.1 μm and about 600 μm may be suitable for promoting bone ingrowth. In one embodiment, the average approximate diameter of the pores 20 is between about 0.1 μm and about 600 μm. In this specification, pore and aperture may be used interchangeably. In some instances, an aperture is an opening in the green tape and a pore or pore structure is the result of connecting apertures in adjacent layers of green tape.

In certain embodiments, the pores 20 may be formed to have different shapes, sizes, and orientations, to provide desired characteristics to the implant 10. For example, the pores 20 may, in certain embodiments, be elongated and oriented to have a substantially unidirectional anisotropy. This configuration may decrease the modulus of elasticity of the stem 14 in a lateral direction 24 while preserving much of the stem's load-bearing capacity in a longitudinal direction 22. In one embodiment, a width of the longitudinal pores is between about 0.1 μm and about 600 μm. In another embodiment, a length of the longitudinal pores is between about 0.1 μm and about 600 μm. The density and length of the pores 20 may also be varied along the length of the implant 10 to vary the modulus of elasticity or strength of the stem 14 at different locations. For example, a neck 26 of the implant 10 may have few if any pores to retain the material's stiffness in that region and because stress shielding in that area may be of little concern.

In other embodiments, elongated pores 20 may be designed to have a varying directional anisotropy. For example, as shown in FIG. 1A, the orientation of the elongated pores changes along the stem 14 to conform to the stem's contour. This technique may be used to change an implant's strength and modulus of elasticity along different axes and may be helpful where an implant is curved or rounded.

Referring to FIG. 1B, one embodiment of a cross-section 27 of the implant 10 of FIG. 1A is illustrated. As shown, in selected embodiments, the pore density may be designed to vary between an outer surface 28 and a core 30 of the stem 14. For example, the pores 20 may be quite dense at or near the outer surface 28. This pore density may decrease moving radially towards the core 30, which may have very few if any pores 20. Because much of the strain may occur at the surface of a support member under load, the higher pore density near the surface 28 may provide greater flexibility to the implant 10 while retaining much of the strength at or near the core 30. In other applications, the reverse approach—higher pore density in the center and low density at the surface—may be more suitable. Accordingly, the apertures may be cut during the cutting step such that the pore density of the implant is greater in an exterior portion than an interior portion. The apertures may also be cut during the cutting step such that the pore density of the implant is greater in an interior portion than an exterior portion. It will be appreciated by those of skill in the art that the formation of apertures cut during the cutting step and layered during the layered step may vary to provide different structural characteristics of the finished implant.

In selected embodiments, the implant 10 illustrated in FIGS. 1A and 1B may be constructed by stacking green metal or ceramic tape in various ways, including, but not limited to, a vertical stack of somewhat circular sheets with circular apertures or a lateral stack of elongated sheets with elongated apertures. Alternatively, a block or mass of layered sheets may be produced with a predetermined pore structure which may then be machined to fit a specific application or use.

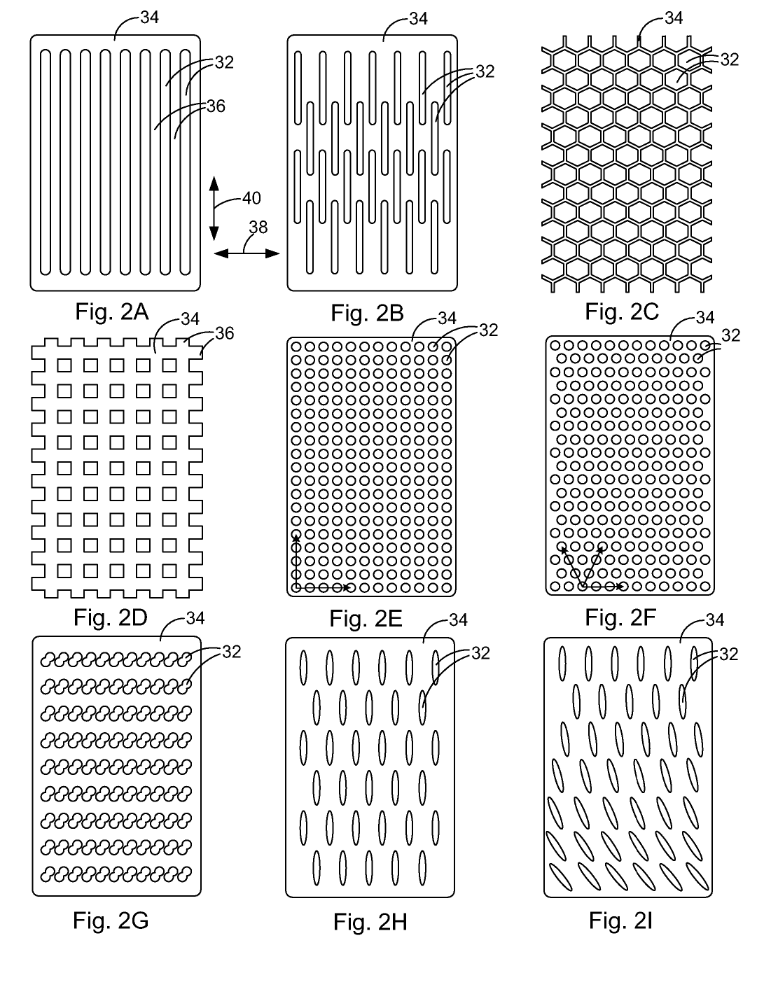

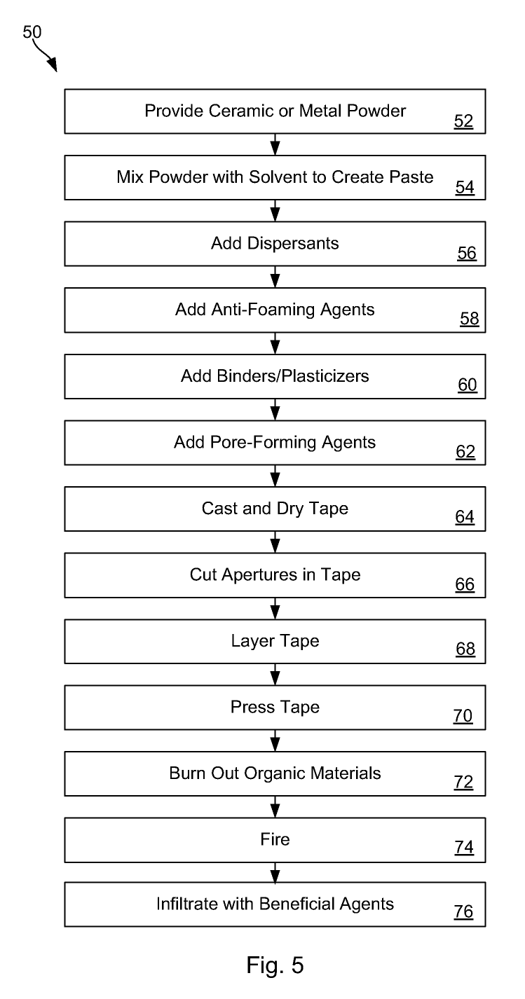

Referring generally to FIGS. 2A through 2I, as will be explained in more detail in association with FIG. 5, an implant 10 having a precisely tailored pore structure may be produced from a layered structure of metal or ceramic green tape. Apertures of various shapes and orientations may be cut in these green tapes to create a desired pore structure in the implant 10. FIGS. 2A through 2I show various embodiments of aperture sizes, orientations, and patterns that may be cut in the green tape to produce different pore structures, each of which may be useful in different applications. These patterns do not represent an exhaustive list, but are simply provided to show examples of various pore structures in accordance with the invention. In one embodiment, the apertures are cut randomly into the green tape. In another embodiment, the apertures are cut into the green tape according to a predetermined pattern.

For example, referring to FIG. 2A, in selected embodiments, elongated apertures 32 may be cut in a layer of green tape 34 to produce elongated pores in an implant 10. Columns 36 of material may remain between each of the apertures 32. Such a configuration may increase the flexibility of the resulting implant structure in a direction 38 relative to the elongated apertures 32, resulting in a structure with a modified modulus of elasticity. However, the columns 36 may continue to provide substantial support in a direction 40.

Similarly, referring to FIG. 2B, in other embodiments elongated apertures 32 may be provided in a staggered configuration, similar to the implant 10 shown in FIG. 1A. Such a configuration may provide additional flexibility in a direction 38 while retaining the ability to bear a substantial load in a direction 40. A staggered pattern may also provide improved load-bearing capacity in a direction 38 compared to the pattern shown in FIG. 2A.

Referring to FIG. 2C, in other embodiments, the tape 34 may be cut into a honeycomb structure forming a network of apertures 32 or geometric cells 32. Honeycomb structures are useful in a wide variety of application due to their high stiffness and low weight. Although of a hexagonal shape in this example, the geometric cells 32 may take on other shapes (e.g., triangles, squares, etc.) as well, although each may have different mechanical properties. In selected embodiments, a honeycomb layer 34 may be sandwiched between less porous layers, such as solid layers, to provide additional rigidity in the plane parallel to the honeycomb layer 34.

Referring to FIG. 2D, in other embodiments, the tape 34 may be cut into a crisscross pattern or other lattice pattern. Such a pattern may be effective to modify an implant's modulus of elasticity while retaining substantial strength and load-bearing capacity along several directions. For example, a crisscross pattern may include columns 36 which are perpendicular to one another. These columns 36 may support significant loads in directions parallel to the columns, providing significant load-bearing capacity in the directions 38, 40. The columns 36 may be oriented, as needed, to support loads from different angles, and do not necessarily need to be oriented perpendicular to one another. Similarly, a crisscross or lattice pattern may include columns 36 that are oriented in more than just two directions.

Referring to FIGS. 2E, 2F, and 2G, in other embodiments, a pattern of circular apertures 32 may be cut in the green tape 34. For example, circular apertures 32 may be arranged in a matrix along two perpendicular axes, as illustrated in FIG. 2E, or along three axes rotated approximately sixty degrees relative to one another, as illustrated in FIG. 2F. Implants 10 implementing these patterns may have different mechanical properties. Similarly, in other embodiments, the circular apertures 32 may be formed such that they interconnect, as illustrated in FIG. 2G. Thus, the pore structure of an implant 10 may be designed to include an interconnected network of pores. As will be explained in more detail hereafter, an interconnected, or open, pore structure may enable various substances to travel through the pore structure of an implant 10. For example, the pore structure of an implant 10 may be infiltrated with beneficial agents that exude into the body around the implant 10.

Referring to FIGS. 2H and 2I, in other embodiments, elongated apertures, such as the elliptically shaped apertures shown, may be designed to have a desired directional anisotropy. This anisotropy may be oriented to give an implant 10 various desired mechanical properties, including load-bearing capacity or flexibility in desired directions. This anisotropy may be substantially unidirectional in some cases, as illustrated in FIG. 2H. The orientation of the anisotropy may also vary in the implant 10. As illustrated in FIG. 2I, the anisotropy of the apertures 32 may change based on their location in the implant 10. This may be useful with implants 10 that are curved, subject to varying loads at different locations, or require different mechanical properties at different locations.

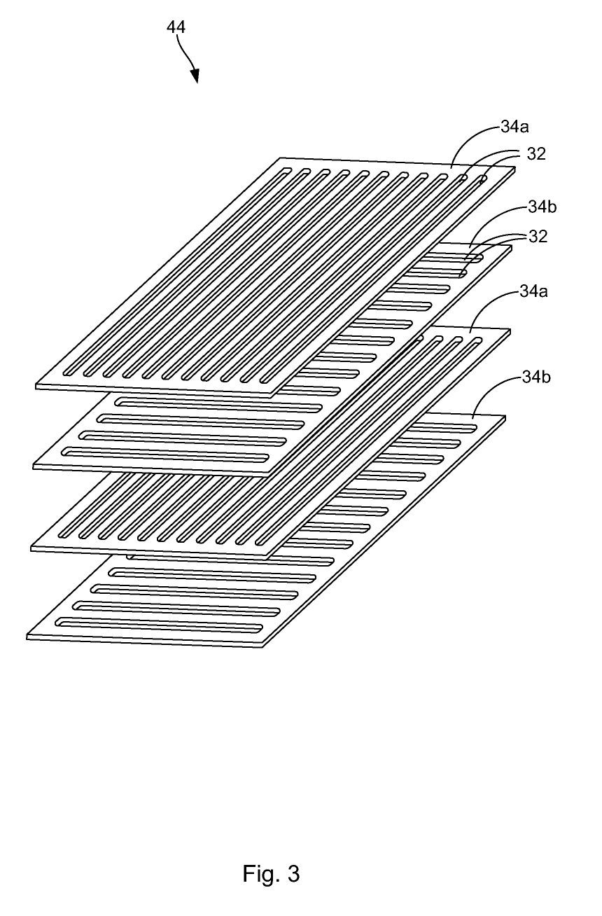

Referring to FIG. 3, in selected embodiments, layers of green tape 34a, 34b may be stacked and oriented in different directions to create a desired pore structure, which includes pore orientation. For example, layers of green tape 34a, 34b may have elongated apertures 32 oriented in different directions, such as perpendicular to one another. These layers 34a, 34b may be stacked to create a crisscross network of apertures 32 and corresponding structural elements to add strength and flexibility to an implant 10 in several directions. Indeed, the layers of green tape 34, including but not limited to those described in association with FIGS. 2A through 2I may be stacked in many different orders, combinations, and orientations to provide desired strength, flexibility, or other mechanical properties.

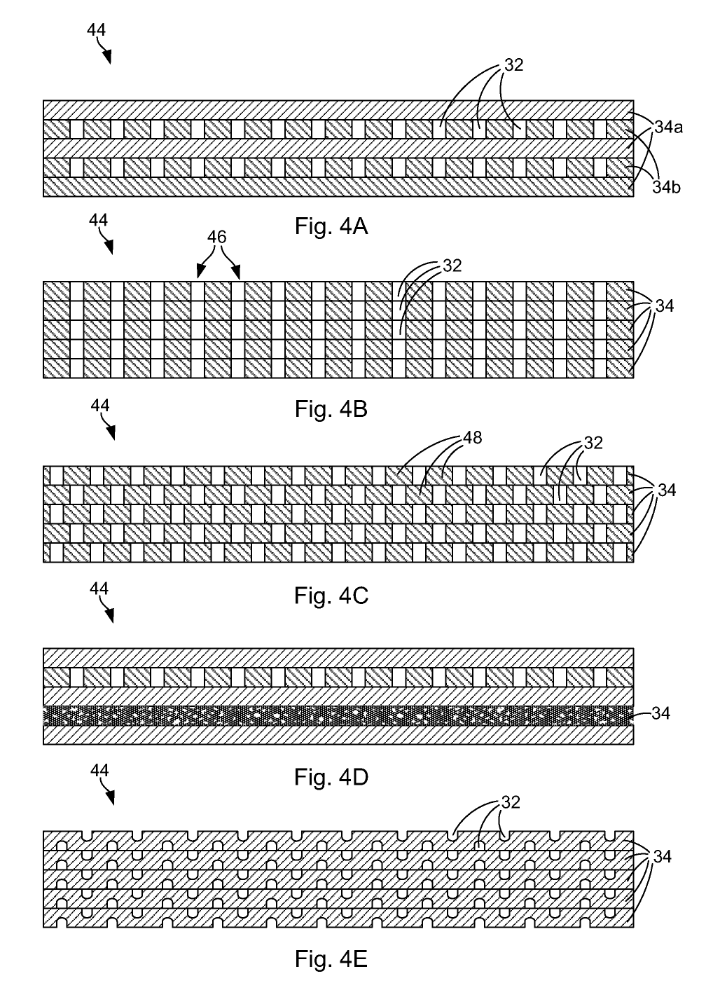

Referring generally to FIGS. 4A through 4E, profile views of various embodiments of layered implant structures are illustrated. These views are simply examples of several contemplated embodiments and are not an exhaustive list of all combinations or orientations that are possible in a layered structure. Indeed, many permutations and combinations of different layers are possible and within the scope of the present invention.

Referring to FIG. 4A, in certain embodiments, a solid implant structure 44 may include multiple layers 34a, 34b. Some layers 34b may include apertures 32 such as those illustrated in FIGS. 2A through 2I while other layers 34a may include few if any apertures 32. For example, honeycomb layers 34b like those described in association with FIG. 2C or other porous layers 34b may be sandwiched between other solid or substantially solid layers 34a. Because some porous layers 34b may be weak on their own, this configuration may add strength to the layered structure 44 while still providing improved flexibility or other desired mechanical properties.

Referring to FIG. 4B, in other embodiments, the layers 34 of green tape of an implant structure 44 may be arranged such that their apertures 32 align or interconnect. This may create channels 46 through the implant 10 and provide an open pore structure. In certain embodiments, these channels 46 may be infiltrated with beneficial agents, such as anti-infective agents or bone growth factors, which may exude from the implant 10 to provide various benefits to the body. The channels 46 may also change the mechanical properties of the implant 10, such as decrease the modulus of elasticity compared to a non-porous implant and increase the modulus of elasticity relative to a porous implant with randomly oriented and randomly distributed porosity.

Referring to FIG. 4C, in other embodiments, layers 34 of green tape may be stacked in a way that apertures 32 are isolated from one another, thereby creating a closed pore structure. An implant 10 with a closed pore structure may, in some cases, be stronger than one with open pores since there may be greater interconnection between solid elements 48 in each layer 34.

Referring to FIG. 4D, in certain embodiments, if desired, one or more layers 34 having a randomly distributed pore structure may be incorporated into an implant 10 in accordance with the invention. As will be explained in more detail hereafter, these pores may be created by mixing a pore-forming agent into the slip used to produce the green tape 34, and then burning out these pore-forming agents to leave the randomly distributed pores.

Referring to FIG. 4E, in other embodiments, apertures 32 may only partially penetrate each layer 34. Each of these apertures 32 may form a closed pore upon being covered by an adjacent layer 34. Thus, in one embodiment, layering comprises positioning two adjacent layers of green tape such that the apertures of one layer do not align with any apertures of the other layer. This configuration may include adjacent layers where only one of layers has apertures or pores. It will be appreciated by those of skill in the art that successive layers may be positioned or layered to provide various multilayered apertures or pores of different configurations and that these configurations may be utilized to achieve a desired characteristic or quality in the implant.

Referring to FIG. 5, one embodiment of a method 50 for making flexible green tapes of metal and ceramic and using these tapes to create an implant 10 is disclosed. It should be recognized that the method 50 may be used to produce green tapes containing a wide variety of chemical compositions, including, but not limited to, any of numerous ceramic powders, metal powders, and mixtures thereof in virtually any ratio. Suitable metal powders may include, for example, powders of iron, aluminum, copper, zinc, tungsten, titanium, tantalum, stainless steel, cobalt, and alloys thereof such as Co—Cr. Metal powders as used herein includes metal alloys including the alloys of the metals described above. Suitable ceramic powders may include, for example, hydroxyapatite, tri-calcium phosphate, titania, zirconia, yttria, alumina, magnesia, calcia, spinel, chromia, perovskites, silicon carbide, silicon nitride, titanium carbide, boron carbide, boron nitride, silica, and the like. It should also be recognized that some embodiments of a method 50 in accordance with the invention may include more, or fewer, steps than those listed in FIG. 5. Thus, one or more steps may be added or deleted from the method 50. The steps listed in FIG. 5 simply represent various steps that may be included in a method 50 in accordance with the invention.

An initial step of a method 50 in accordance with the invention may include providing 52 a ceramic or metal powder, or mixture thereof, such as one of the powders listed above. This powder may then be mixed 54 with an aqueous or non-aqueous solvent to form a mixture. In one embodiment, the mixture is a paste. Suitable solvents may include but are not limited to water, methanol, acetone, ethanol, isopropyl alcohol, butanol, toluene, xylene, hexanol, methyl ethyl ketone (MEK), hexane, or mixtures thereof. In certain embodiments, the solvent may comprise about two to ninety percent of the total volume. In other embodiments, water may comprise about ten to sixty percent of the total volume. In still other embodiments, water may comprise about twenty to twenty-five percent of the total volume.

In selected embodiments, a suitable dispersant may be added 56 to provide a lower viscosity suspension if desired. Suitable dispersants may include, for example, ammonium polymethacrylate (Darvan), polymethyl methacrylate (PMMA), glycerol, polyvinyl butyral (PVB), polyvinyl alcohol (PVA), or other suitable dispersants known to those of skill in the art. In some cases, the dispersant may comprise about 0.001 to about 10 percent of the total weight of the mixture. A suitable anti-foaming agent may also be added 58 to create a non-foaming suspension if desired. Such anti-foaming agents may include, for example, Triton® X-100. In some instances, the anti-foaming agent may comprise about 0.001 to about 10 percent of the total weight.

After a homogeneous mix is obtained, binders and plasticizers may be added 60 to the mixture. Suitable binders and plasticizers may include, for example, PVA, PVB, Santicizer® 160, dibutyl phthalate (DBP), glycerol, or the like. In selected embodiments, the binder may comprise about 0.01 to about 25 percent of the total weight. Similarly, a plasticizer may comprise about 0.01 to about 25 percent of the total weight.

If a random pore structure, such as that illustrated in FIG. 4D, is desired, a pore-forming agent may also be added 62 to the mixture. For example, suitable pore-forming agents may include but are not limited to one or more of microcellulose, agar, flour, salt, sugar, polymer beads, polymer powder, polymer fiber, oil, chopped hair, paper, wood chips, carbon, burnt ash, PVA, PVB, glycerol, or other organic materials known to those of skill in the art. These pore-forming agents may comprise about 0.001 to about 90 percent of the total weight of the mixture.

The foregoing steps may be used to prepare a final homogeneous suspension or slip. The slip may then be cast 64 into thin sheets by spreading the slip onto a substrate to create a film. Suitable substrates may include, for example, surfaces of glass, plastic, wood, metal, Mylar®, paper, or the like. The slip may then be spread manually, such as using a doctor blade, or in an automated process using a table-top, research, or production-type tape caster or by using other methods known to those of skill in the art. In selected embodiments, the thickness of the wet film may vary between about 0.001 mm and 100 mm. In other embodiments, the thickness may vary between about 0.01 mm and 0.1 mm. The wet film may then be dried 64 at a temperature ranging from about 0° C. to about 100° C. in an open lab (i.e., at room temperature), a temperature controlled oven, or in the heating zone of an automated tape caster.

After drying 64, the film may then be removed from the substrate and apertures 32 may be cut 66 into the dry green tape. These apertures 32 may be cut using a wide variety of techniques including but not limited to laser cutting, etching, mechanical cutting, and burning. For example, mechanical cutting may be performed by manual or automated operation of a blade, punch, drill bit, or other cutting device. A laser cutter may be used to provide additional accuracy. The apertures 32 may be cut in any shape or size, and may be produced as patterns of identical or mixed shapes. In certain embodiments, the aspect ratio of the apertures may range from about 0.001 to about 1000. In other embodiments, the aspect ratio of the apertures may range from about 20 to about 500.

Once the apertures 32 are formed, the green tape may be layered 68 (i.e., stacked and oriented) to create a desired pore structure. The layered tape may then be subjected to a process wherein they are fused together. The fusing step may include laminating the layers of green tape. The fusing step may also include firing or sintering the layers of green tape. In one embodiment, the fusing step includes sintering laminated layers of green tape. When fusing includes pressing 70 the layers together to form a laminated structure, the pressure applied may, in certain embodiments, range from about 1 PSI to about 150,000 PSI. The pressure applied may also be tailored to the specific materials used, the pore size, density, and shape within the laminated structure, the final shape of the laminated structure, or the like. In selected embodiments, pressing 70 may occur at a temperature between about 0° C. and 100° C.

After pressing, the laminated structure may then be subjected 72 to an organic bum-out cycle to remove all or a substantial part of the organic constituents in the tape, including but not limited to the pore-forming agents discussed above. In certain embodiments, the organic burn-out temperature may range from about 20° C. to about 1000° C. After this bum-out process 72 is complete, the structure may be fired 74 at higher temperatures to achieve a desired strength. In certain embodiments, these firing or sintering temperatures may range from about 100° C. to about 2300° C.

Once firing is complete, the user if left with a hardened load-bearing implant structure having the desired pore structure. As previous mentioned, this pore structure may, in certain embodiments, create channels or cavities in the implant structure. This pore structure or channel structure or cavity structure may be used to control or adjust the modulus of elasticity, the flexural strength, or the porosity of the implant structure.

If desired, these channels or cavities may be infiltrated 76 with one or more beneficial agents for delivery to the body upon implantation. Such beneficial agents may be used, for example, to prevent or reduce infection or inflammation, or to promote bone ingrowth, healing, osteoconduction, osteointegration, drug delivery, or the like. Beneficial agents may include but are not limited to bone growth factors, bone morphogenic proteins, hydroxyapatite, tricalcium phosphate, osteoconducting elements and compounds, collagen fibers, blood cells, bone cements, osteoblast cells, antibiotic agents, anti-bacterial agents, anti-inflammatory agents, cancer drugs, pain-relieving drugs, and the like.

The following are several non-limiting examples of implant structures created using a method 50 in accordance with the invention:

### EXAMPLE 1

In a first example, ceramic alumina powder is mixed with water with the powder comprising about twenty to twenty-five percent of the total volume. A dispersant comprising less than one percent of the total weight is added to the mixture. PVA and glycerin are added to the mixture in a ratio of about 2:1 to obtain a final slip. This slip is then tape cast to a thickness of about 10 mils (0.01 inches) and dried to obtain a flexible tape. The tape is then laser cut to obtain channels like those illustrated in FIG. 2A.

### EXAMPLE 2

In a second example, ceramic perovskite powder is mixed with toluene-ethanol mixtures with the ceramic powder comprising about forty to fifty percent of the total volume. A dispersant comprising less than one percent of the total weight is added to the mixture. PVB and Santicizer® 160 are added to the mixture in a ratio of about 2:1 to obtain a final slip. This slip is then tape cast to a thickness of about 10 mils (0.01 inches) and dried to obtain a flexible tape. The tape is then laser cut to obtain channels in the tape. The individual laser cut sheets are then stacked and laminated at a pressure of less than about 10,000 PSI at 60° C. The green laminated structure is then fired to between about 1200° C. to 1500° C. to obtain a fired component that has a tailored pore structure built into the device.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. a method producing implants, the method comprising:
providing a green tape comprising at least one of metal and ceramic particles for incorporation into a solid implant structure;
cutting apertures in selected regions of the green tape in order to create a desired pore structure in the solid implant structure;
layering the green tape in an orientation that will provide the desired pore structure; and
fusing a plurality of layers together to create the solid implant structure with the desired pore structure.

2. The method of claim 1, wherein the green tape comprises metal particles.

3. The method of claim 2, wherein the metal particles comprise one of the group consisting of: powders of iron, aluminum, copper, zinc, tungsten, titanium, tantalum, stainless steel, cobalt, and combinations thereof.

4. The method of claim 1, wherein the green tape comprises ceramic particles.

5. The method of claim 4, wherein the ceramic particles comprise one of the group consisting of: hydroxyapatite, tri-calcium phosphate, titania, zirconia, yttria, alumina, magnesia, calcia, spinel, chromia, perovskites, silicon carbide, silicon nitride, titanium carbide, boron carbide, boron nitride, silica, and combinations thereof.

6. The method of claim 1, wherein providing a green tape further comprises the steps of mixing a ceramic or metal powder with an aqueous or non-aqueous solvent to form a mixture.

7. The method of claim 6, wherein the solvent comprises on of the group consisting of: water, methanol, acetone, ethanol, isopropyl alcohol, butanol, toluene, xylene, hexanol, methyl ethyl ketone (MEK), hexane, or mixtures thereof.

8. The method of claim 6, wherein providing a green tape further comprises adding a dispersant to the mixture.

9. The method of claim 8, wherein the dispersant may comprise about 0.001 to about 10 percent of the total weight of the mixture.

10. The method of claim 6, wherein the mixture comprises a plasticizer.

11. The method of claim 6, wherein the mixture comprises a binder.

12. The method of claim 1, wherein providing a green tape further comprises spreading a slip onto a substrate.

13. The method of claim 1, wherein cutting further comprises at least one of laser cutting, etching, mechanical cutting, and burning apertures in the green tape.

14. The method of claim 1, wherein the apertures comprise an aspect ratio of between about 0.001 to about 1000.

15. The method of claim 1, wherein the apertures comprise an aspect ratio of between about 20 to about 500.

16. The method of claim 1, wherein the apertures comprise a diameter of between about 0.1 microns and about 600 microns.

17. The method of claim 1, wherein the apertures are cut such that the pore density of the implant is greater in an exterior portion than an interior portion.

18. The method of claim 1, wherein the apertures are cut such that the pore density of the implant is greater in an interior portion than an exterior portion.

19. The method of claim 1, wherein cutting further comprises cutting elongated apertures.

20. The method of claim 19, wherein cutting elongated apertures further comprises cutting elongated apertures with a desired directional anisotropy.

21. The method of claim 1, wherein the apertures are randomly cut into the green tape.

22. The method of claim 1, wherein the apertures are cut into the green tape according to a predetermined pattern.

23. The method of claim 1, wherein the pore structure comprises at least one of a plurality of interconnected pores and a plurality of closed pores.

24. The method of claim 1, wherein pores of the pore structure are sized to promote bone ingrowth into the pore structure.

25. The method of claim 1, wherein the pore structure is characterized by at least one of pore density, pore orientation, pore spacing, and pore spatial location, the pore structure varying along at least one a radial direction, length, width, and height of the solid implant structure.

26. The method of claim 1, wherein layering comprising layering two adjacent layers of green tape such that the apertures of one layer do not align with any apertures of the other layer.

27. The method of claim 1, wherein fusing further comprises pressing the layered green tapes together to form a laminated structure.

28. The method of claim 27, wherein pressing is accomplished between about 1 and about 150,000 pounds per square inch.

29. The method of claim 27, wherein fusing further comprises firing the laminated structure to burn off organic materials in the laminated structure.

29. The method of claim 27, wherein fusing further comprises sintering the laminated structure.

30. The method of claim 29, wherein the laminated structure is sintered at a temperature between about 100 degrees Celsius and about 2300 degrees Celsius.

31. The method of claim 1, further comprising infiltrating the pore structure with beneficial agents, wherein the beneficial agents are selected from the group consisting of bone growth factors, bone morphogenic proteins, hydroxyapatite, calcium sulfate, tricalcium phosphate, osteoconducting elements and compounds, collagen fibers, blood cells, bone cements, osteoblast cells, antibiotic agents, anti-bacterial agents, anti-inflammatory agents, cancer drugs, and pain-relieving drugs.

32. An implant produced by the steps of:
providing a green tape comprising at least one of metal and ceramic particles for incorporation into a solid implant structure;
cutting apertures in selected regions of the green tape in order to create a desired pore structure in the solid implant structure;
layering the green tape in an orientation that will provide the desired pore structure; and
fusing a plurality of layers together to create the solid implant structure with the desired pore structure.

33. The implant of claim 32, wherein cutting further comprises at least one of laser cutting, etching, mechanical cutting, and burning apertures in the green tape.

34. The implant of claim 32, wherein cutting further comprises cutting elongated apertures.

35. The implant of claim 34, wherein cutting elongated apertures further comprises cutting elongated apertures with a desired directional anisotropy.

36. The implant of claim 32, wherein the pore structure comprises at least one of a plurality of interconnected pores and a plurality of closed pores.

37. The implant of claim 32, wherein pores of the pore structure are sized to promote bone ingrowth into the pore structure.

38. The implant of claim 32, wherein the pore structure is characterized by at least one of pore density, pore orientation, pore spacing, and pore spatial location, the pore structure varying along at least one a radial direction, length, width, and height of the solid implant structure.

39. The implant of claim 32, wherein fusing further comprises pressing the layered green tape together to form a laminated structure.

40. The implant of claim 39, wherein fusing further comprises firing the laminated structure to burn off organic materials in the laminated structure.

41. The implant of claim 40, wherein fusing further comprises sintering the laminated structure.

42. The implant of claim 32, further produced by the step of infiltrating the pore structure with beneficial agents.

43. An implant comprising:
a solid implant structure comprising a plurality of layers fused together, the layers comprising at least one of metal and ceramic particles fused together, the layers further comprising apertures cut therein to provide a desired pore structure in the solid implant structure, the pore structure designed such to provide at least one of a desired modulus of elasticity, flexural strength, and porosity to the solid implant structure.

44. The implant of claim 43, wherein the apertures are elongated apertures.

45. The implant of claim 44, wherein the elongated apertures are characterized by directional anisotropy.

46. The implant of claim 43, wherein the pore structure comprises at least one of a plurality of interconnected pores and a plurality of closed pores.

47. The implant of claim 43, wherein pores of the pore structure are sized to promote bone ingrowth into the pore structure.

48. The implant of claim 43, wherein the pore structure is characterized by at least one of pore density, pore orientation, pore spacing, and pore spatial location, the pore structure varying along at least one a radial direction, length, width, and height of the solid implant structure.

49. The implant of claim 43, wherein the pore structure is infiltrated with beneficial agents.

50. The implant of claim 43, wherein the plurality of layers are selected from the group consisting all metal layers, all ceramic layers, a combination of metal and ceramic layers, a combination of layers of different ceramic materials, a combination of layers of different metals, and combinations thereof.

51. The implant of claim 43, wherein the plurality of layers comprises layers having different pore structures.
